(19) United States
(12) Patent Application Publication
(10) Pub. No.: US 2007/0281988 A1
(43) Pub. Date: Dec. 6, 2007

(54) Combination Therapy for Vascular Complications Associated with Hyperglycemia

(76) Inventors: Norman Eugene Cameron, Aberdeen (GB); Hans-Jurgen Mest, Hamburg (DE); Klaus Seedorf, Hamburg (DE); Luitgard Seedorf, Hamburg (DE)

Correspondence Address: ELI LILLY & COMPANY, PATENT DIVISION, P.O. BOX 6288, INDIANAPOLIS, IN, 46206-6288, US

(21) Appl. No.: 11/720,087

(22) PCT Filed: Dec. 16, 2005
(86) PCT No.: PCT/US05/45865

§ 371 Date: May 24, 2007

Related U.S. Application Data
(60) Provisional application No. 60/637,527, filed on Dec. 20, 2004.

(51) Int. Cl. A61K 31/35; A61K 31/40; A61K 31/405; A61P 27/00; A61P 9/10

(52) U.S. Cl. 514/415; 514/410; 514/460

(57) ABSTRACT

The present invention relates to a method of treating one or more vascular complications of hyperglycemia comprising administering to a patient in need of treatment for one or more vascular complications to hyperglycemia a therapeutically effective amount of ruboxistaurin, or a pharmaceutically acceptable salt thereof in combination with a therapeutically effective amount of an HMG-CoA reductase inhibitor.

### BACKGROUND OF THE INVENTION

Diabetes mellitus is a global health problem, affecting all age groups. Currently, around 177 million people have diabetes worldwide; however, the World Health Organization (WHO) projects that this number will increase to at least 300 million by 2025. The diabetic epidemic relates in particular to Type 2 diabetes, which accounts for around 90% of all diabetes cases. The increased prevalence of Type 2 diabetes can be attributed to the aging population and rising incidence of obesity in the developed countries, among other factors.

Prevention of complications specific to diabetes is a key issue because of the morbidity and mortality associated with the disease. Clinically significant morbidity may often develop before diagnosis. Although not everyone with diabetes will develop a complication, a recent epidemiological study reported that two or more complications are apparent in almost one fifth of people with diabetes. Morgan C L, Currie C J, Stott N C H et al.; “The prevalence of multiple diabetes-related complications.” Diabet Med 17:146-151 (2000).

Microvascular complications develop in most people with Type 1 and Type 2 diabetes and are associated with clinically significant morbidity and mortality. It has been suggested that subsets of patients with Type 1 diabetes may have a genetically determined susceptibility to microvascular complications as not all people with Type 1 diabetes and very high blood glucose levels develop complications. Conversely, some develop complications even if blood glucose levels are only slightly elevated. The prevalence of Type 2 diabetes is increasing across all ethnic groups, particularly among black and minority groups. Because Type 2 diabetes is often not diagnosed until the patient has had the disease for many years, long-term complications may be present at the time diabetes is discovered.

Although there are several known risk factors, chronic hyperglycemia is a major initiator of certain microvascular complications of diabetes such as diabetic retinopathy, nephropathy and neuropathy. The landmark Diabetes Control and Complications Trial (DCCT) has shown that the more time individuals are exposed to chronically elevated plasma glucose levels, the greater their risk of microvascular complications. The Diabetes Control and Complications Trial Research Group; “The effect of intensive treatment of diabetes on the development and progression of long-term complications in insulin-dependent diabetes mellitus;” N. Eng. J. Med 329:977-986 (1993). In addition, the deleterious effects of hyperglycemia on the microcirculation have been shown to persist for a considerable time after glucose levels have decreased. Both the DCCT and another landmark study, the United Kingdom Prospective Diabetes Study group (UKPDS), have shown that intensive glycemic management slows the progression of microvascular complications in Type 1 and Type 2 diabetes, and thereby improves quality of life. Turner R, Holman R, Stratton I et al.; “Tight blood pressure control and risk of macrovascular and microvascular complications in type 2 diabetes: UKPDS 38;” BMJ 317:703-713 (1998). Although intensive therapy may adversely affect the development of retinopathy, the DCCT concluded that the long-term benefits of intensive insulin therapy greatly outweigh the early risks of retinopathy. The Diabetes Control and Complications Trial Research Group. “Early worsening of diabetic retinopathy in the Diabetes Control and Complications Trial.” Arch. Opthalmol. 116:874-886 (1998).

Despite good long-term glycaemic and blood pressure control, diabetes remains a major cause of blindness, renal failure and amputations, all of which result in significant health care expenditure. As the incidence of diabetes continues to rise, the burden of vascular complications will increase in the future.

### BRIEF SUMMARY OF THE INVENTION

The present invention relates to combination methods for treating vascular complications to hyperglycemia including (1) microvascular complications, such as diabetic peripheral neuropathy, diabetic retinopathy, diabetic macular edema, and diabetic nephropathy; and (2) macrovascular complications, such as cardiovascular disorders comprising congestive heart failure, atherosclerosis, cerebrovascular disease, and hypertension.

More specifically, the present invention relates to a method of treating one or more vascular complications to hyperglycemia comprising administering to a patient in need of said treatment a therapeutically effective amount of ruboxistaurin, or a pharmaceutically acceptable salt thereof in combination with a therapeutically effective amount of an HMG-CoA reductase inhibitor.

The present invention also relates to a method for achieving a synergistic therapeutic effect comprising treating one or more vascular complications of hyperglycemia in a patient in need thereof which comprises administering to said patient synergistic effective amounts of:
(a) ruboxistaurin or a pharmaceutically acceptable salt thereof; and
(b) an HMG-CoA reductase inhibitor selected from the group consisting essentially of lovastatin, simvastatin, pravastatin, fluvastatin, atorvastatin, rivastatin, or a pharmaceutically acceptable salt thereof;

wherein the amount of (a) alone and the amount of (b) alone is less than an amount indicated to achieve the maximal therapeutic effect; and wherein the combined effect of the amounts of (a) and (b) administered is greater than the sum of the therapeutic effects of the amounts of (a) and (b) individually administered.

The present invention further relates to a pharmaceutical formulation comprising ruboxistaurin, or a pharmaceutically acceptable salt thereof; an HMG-CoA reductase inhibitor; and a pharmaceutical carrier, diluent, or excipient.

### DETAILED DESCRIPTION OF THE INVENTION

As used herein, the term “vascular complications to hyperglycemia” includes macrovascular complications to hyperglycemia and microvascular complications to hyperglycemia.

“Macrovascular complications to hyperglycemia” refers to any complication of diabetes mellitus or non-diabetic hyperglycemia that is due to a macrovascular mediated cause which includes: peripheral vascular disease (affecting the blood vessels outside the heart and brain and is often a narrowing of vessels that carry blood to leg and arm muscles), cerebrovascular disease (referring to conditions of the blood vessels of the brain, including stroke, cerebral arteriosclerosis, cerebral aneurysm and cerebral artery disease), diabetic cardiovascular disease (the leading cause of premature death among people with diabetes) and hypertension.

“Microvascular complications to hyperglycemia” refers to any complication of diabetes mellitus or non-diabetic hyperglycemia that is due to a microvascular mediated cause which includes: diabetic eye disease (including retinopathy, macular edema, blindness), diabetic nerve disease (including neuropathy, autonomic neuropathy, foot ulceration, amputation), and diabetic kidney disease (including microalbuminuria, proteinuria, nephropathy, end-stage renal disease, hemodialysis).

Ruboxistaurin is also known as: (S)-9-((Dimethylamino)methyl)-6,7,10,11-tetrahydro-9H,18H-5,21:12,17-dimethenodibenzo(e,k)pyrrolo(3,4-h)(1,4,13)oxadiaza-cyclohexadecine-18,20(19H)-dione. Ruboxistaurin mesylate monohydrate is currently in Phase III clinical trials for various microvascular complications to diabetes and is structurally depicted as: 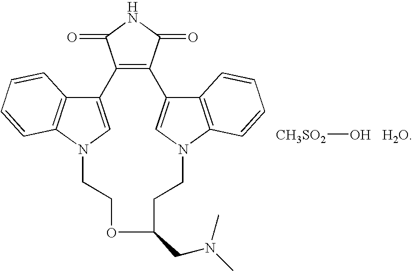
Ruboxistaurin, its pharmaceutically acceptable salts, and related compounds are described in Heath, Jr. et al., U.S. Pat. No. 5,552,396. The mesylate salts of ruboxistaurin are specifically described and claimed in U.S. Pat. No. 5,710,145. The synthesis of ruboxistaurin, its salts and related compounds as well as a disclosure that said compounds are useful in the treatment of conditions associated with diabetes mellitus and its complications as well as ischemia, inflammation, central nervous system disorders, cardiovascular disease, dermatological disease, Alzheimer's disease and cancer. The use of ruboxistaurin in treating vascular endothelial cell dysfunction, microalbuminuria, cardiovascular disease, central ischemic brain injury, restenosis, atherosclerosis, congestive heart failure, myocardial infarction, and the like, is taught in U.S. Pat. No. 5,723,456. U.S. Pat. Nos. 5,552,396, 5,710,145, and 5,723,456 are hereby incorporated by reference in their entirety as if fully set forth.

Compounds which have inhibitory activity for HMG-CoA reductase can be readily identified by using assays well-known in the art. For example, see the assays described or cited in U.S. Pat. No. 4,231,938 at col. 6, and WO 84/02131 at pp. 30-33, both references incorporated by reference herein as if fully set forth. The term HMG-CoA reductase inhibitor is intended to include all pharmaceutically acceptable salts of compounds which have HMG-CoA reductase inhibitory activity, and therefore the use of such salts is included within the scope of this invention.

Examples of HMG-CoA reductase inhibitors that may be used include, but are not limited to lovastatin (MEVACOR®), simvastatin (ZOCOR®), pravastatin (PRAVACHOL®), fluvastatin (LESCOL®), atorvastatin (LIPITOR®) and rivastatin (also known as cerivastatin). The structural formulae of these and additional HMG-CoA reductase inhibitors that may be used in the instant methods are described at page 87 of M. Yalpani, “Cholesterol Lowering Drugs”, Chemistry & Industry, pp. 85-89 (Feb. 5, 1996). Prefereably, the HMG-CoA reductase inhibitor is selected from lovastatin and simvastatin.

The term “pharmaceutically-acceptable salt” as used herein, refers to a salt of a ruboxistaurin and/or the HMG-CoA reductase inhibitors herein disclosed. It should be recognized that the particular counterion forming a part of any salt relevant to this invention is usually not of a critical nature, so long as the salt as a whole is pharmacologically acceptable and as long as the counterion does not contribute undesired qualities to the salt as a whole.

Such salts include the pharmaceutically acceptable salts listed in Journal of Pharmaceutical Science, 66, 2-19 (1977), which are known to the skilled artisan. See also, The Handbook of Pharmaceutical Salts; Properties, Selection, and Use. P. H. Stahl and C. G. Wermuth (ED.s), Verlag, Zurich (Switzerland) 2002.

As used herein, the term “patient” refers to a warm-blooded animal or mammal which is in need of treating one or more diabetic vascular complications. It is understood that guinea pigs, dogs, cats, rats, mice, hamsters, and primates, including humans, are examples of patients within the scope of the meaning of the term. A most preferred patient is a human.

As used herein, the term “treating” is defined to include its generally accepted meaning which includes preventing, prohibiting, restraining, and slowing, stopping or reversing progression, or severity, and holding in check and/or treating existing characteristics. The present method includes both medical therapeutic and/or prophylactic treatment, as appropriate.

The term “synergistic” as used herein means that the effect achieved with the methods and compositions of this invention is greater than the sum of the effects that result from methods and compositions comprising the inhibitors and antagonists of this invention separately and in the amounts employed in the methods and compositions hereof.

The instant method involves the administration of ruboxistaurin, or a pharmaceutically acceptable salt thereof, in combination with an HMG-CoA reductase inhibitor. This combination therapy includes administration of a single pharmaceutical dosage formulation which contains both ruboxistaurin, or a pharmaceutically acceptable salt thereof, and the HMG-CoA reductase inhibitor, as well as administration of each active agent in its own separate pharmaceutical dosage formulation. Where the separate dosage formulations are used, ruboxistaurin, or a salt thereof, and the HMG-CoA reductase inhibitor can be administered at essentially the same time, i.e., concurrently, or at separately staggered times, i.e., sequentially.

As used herein, the term “therapeutically effective amount” means an amount of ruboxistaurin, or a salt thereof or an amount of a HMG-CoA reductase inhibitor capable of alleviating the symptoms of the various pathological conditions herein described when administered in combination with one another as herein described. The specific dose of a compound administered according to this invention will, of course, be determined by the particular circumstances surrounding the case including, for example, the particular compounds administered, the route of administration, the state of being of the patient, and the pathological condition being treated.

For ruboxistaurin, or a pharmaceutically acceptable salt thereof, a typical daily dose for human use will contain a nontoxic dosage level of from about 1 to about 1000 mg/day of a compound of the present invention. Preferred daily doses generally will be from about 5 to about 600 mg/day. For ruboxistaurin mesylate, the more preferred doses range from 32 mg to about 128 mg, administered once per day for ruboxistaurin mesylate. The most preferred dose is 39.8 mg of ruboxistaurin mesylate monohydrate (32 mg of ruboxistaurin free base) once per day.

The daily dosage amounts of the HMG-CoA reductase inhibitor are intended to be the same or similar to those amounts which are employed for anti-hypercholesterolemic treatment and which are described in the Physicians' Desk Reference (PDR). For example, see the 50th Ed. Of the PDR, 1996 (Medical Economics Co.); in particular, see at page 216 the heading “Hypolipidemics,” sub-heading “HMG-CoA Reductase Inhibitors,” and the reference pages cited therein. Preferably, the oral dosage amount of HMG-CoA reductase inhibitors is from about 1 to 200 mg/day, and more preferably from about 5 to 160 mg/day. However, dosage amounts will vary depending on the potency of the specific HMG-CoA reductase inhibitor used as well as other factors as noted above. An HMG-CoA reductase inhibitor which has sufficiently greater potency may be given in sub-milligram daily dosages.

As examples, the daily dosage amount for simvastatin may be selected from 5 mg, 10 mg, 20 mg, 40 mg, 80 mg, and 160 mg; for lovastatin, 10 mg, 20 mg, 40 mg, and 80 mg; for fluvastatin sodium, 20 mg, 40 mg, and 80 mg; and for pravastatin sodium, 10 mg, 20 mg, and 40 mg.

The dosage or dosages which will result in optimal synergistic effects is achieved by coordinating the pharmacokinetic properties, such as volume of distribution and Tₘₐₓ, of the therapeutic agents of this in invention so that the therapeutic windows of each agent overlap to the maximum extent possible. Such dosages are readily determined by one skilled in the art enabled by the disclosure herein.

The methods of the present invention can be practiced by administering the claimed combinations alone or in the form of a pharmaceutical composition, that is, combined with pharmaceutically acceptable carriers or excipients, the proportion and nature of which are determined by the solubility and chemical properties of the compounds selected, the chosen route of administration, and standard pharmaceutical practice.

The methods of the present invention can be practiced by administering the claimed combinations orally, by inhalation, or by the subcutaneous, intramuscular, intravenous, transdermal, intranasal, rectal, occular, topical, sublingual, buccal, or other routes. Oral administration is generally preferred for treatment of the disorders described herein. However, oral administration is not the only preferred route. For example, the intravenous route may be preferred as a matter of convenience or to avoid potential complications related to oral administration.

One skilled in the art of preparing formulations can readily select the proper form and mode of administration depending upon the particular characteristics of the active compounds selected, the disorder or condition to be treated, the stage of the disorder or condition, and other relevant circumstances. (Remington's Pharmaceutical Sciences, 18th Edition, Mack Publishing Co. (1990)).

The pharmaceutical compositions relevant to the combination methods disclosed herein are prepared in a manner well known in the pharmaceutical art. The carrier or excipient may be a solid, semi-solid, or liquid material that can serve as a vehicle or medium for the active ingredient. Suitable carriers or excipients are well known in the art. The pharmaceutical composition may be adapted for oral, inhalation, parenteral, or topical use and may be administered to the patient in the form of tablets, capsules, aerosols, inhalants, suppositories, solutions, suspensions, or the like.

### Test Procedures

Diabetes Induction and Experimental Design

Diabetes is induced in mature (19 week old) male Sprague-Dawley by streptozotocin injection (40-45 mg/kg i.p.). The diabetic state is monitored weekly using commercially available test strips for blood (tail vein) and urine glucose levels. Body weight is also be monitored daily. The criteria for the diabetic state are; blood glucose>19.9 mM, glycosuria, and no evidence of body weight gain. After 6 weeks of untreated diabetes, drugs are administered for a 2-week period.

Methods

Nerve Conduction Velocity

In final experiments, rats are anaesthetized with thiobutabarbital (50-100 mg kg⁻¹ i.p.). The trachea is cannulated for artificial respiration. As described in Cameron N E, Cotter M A, Robertson S (1989), “The effect of aldose reductase inhibition on the pattern of nerve conduction deficits in diabetic rats.” Q. J. Exp. Physiol. 74:917-926; and Cameron N E, Cotter M A, Robertson S (1991), “Effects of essential fatty acid supplementation on peripheral nerve and skeletal muscle function and capillarization in streptozotocin diabetic rats.” Diabetes 40:532-539, the sciatic nerve is exposed between the sciatic notch and knee. Bipolar stimulating electrodes are placed close to the nerve at the notch and knee. A concentric bipolar electrode is inserted into tibialis anterior muscle to monitor evoked electromyographic (EMG) activity. Potentials from each stimulating site are averaged 8 times. Motor conduction velocity is calculated by dividing the distance between stimulating electrodes by the average latency difference between the onset of EMG potentials evoked from the 2 sites. Nerve temperature is monitored using a thermocouple probe, and maintained in the range 36-38° C. by radiant heat. Body temperature is also maintained around 37° C. using a heated blanket. Sensory conduction velocity is measured for sensory saphenous nerve between groin and mid calf in a similar fashion, except that direct nerve evoked potentials are recorded at the ankle using a unipolar platinum hook electrode.

Sciatic Endoneurial Blood Flow

Sciatic endoneurial blood flow is estimated in the limb contralateral to that for conduction velocity measurements by microelectrode polarography and hydrogen clearance. Day T J, Lagerlund T D, Low P A (1989), “Analysis of H₂ clearance curves used to measure blood flow in rat sciatic nerve,” J Physiol 414:35-54; Cameron N E, Cotter M A, Low P A (1991), “Nerve blood flow in early experimental diabetes in rats: relation to conduction deficits.” Am J Physiol 261:E1-E8; Cameron N E, Cotter M A, Hohman T C (1996), “Interactions between essential fatty acid, prostanoid, polyol pathway and nitric oxide mechanisms in the neurovascular deficit of diabetic rats,” Diabetologia 39:172-182. Rats are artificially ventilated. The carotid artery is cannulated to monitor blood pressure. The level of anaesthesia is monitored by observing any reaction of blood pressure to manipulation, and supplementary thiobutabarbital anaesthetic is given as necessary. The sciatic nerve is exposed and the skin around the incision sutured to a metal ring to form a pool filled with mineral oil at 37° C. During recordings, pool temperature is maintained at 35-37° C. by radiant heat. A glass-insulated platinum microelectrode, polarized at 250 mV with respect to a subcutaneous reference electrode, is inserted into the sciatic nerve endoneurium between the sciatic notch and the nerve trifurcation above the knee. 10% H₂ would be added to the inspired gas, the proportions of O₂ and N₂ being adjusted to 20% and 70% respectively. When the H₂ current recorded by the electrode has stabilized, indicating equilibrium with arterial blood, the H₂ supply is shut off and N₂ delivery increased appropriately. H₂ clearance is recorded until a stable baseline is reached, which is defined as no systematic decline in electrode current over 5 min. This procedure is then repeated at another nerve site. After the experiment, mono- or bi-exponential curves are fitted to the clearance data by computer using non-linear regression analysis (Prism, Graphpad, San Diego, Calif., USA) and the general bi-exponential equation:
y=aexp(−bx)+cexp(−dx)+e
Where y is the electrode hydrogen current (arbitrary units), x is time (min), a and c are weighting constants for fast (non-nutritive) and slow (nutritive) clearance components respectively, b is the fast component and d is the slow component (ml min⁻¹ ml nerve⁻¹), and e is the baseline electrode current (arbitrary units). Assuming a tissue density of 1, nutritive blood flow would be calculated as d×100 (ml min⁻¹ 100 g⁻¹). Vascular conductance is calculated by dividing blood flow by the mean arterial blood pressure over the recording period for that particular clearance curve. The averages from the two determinations are taken to represent sciatic endoneurial blood flow parameters.
Results

Interactions between ruboxistaurin and lovastatin: Dose ranging studies were first conducted to find an appropriate dose of lovastatin around the ED₂₀ for motor conduction velocity. As set forth in the Table below, the diabetic deficits in motor and sensory conduction velocity were partially (˜20%) corrected by the individual doses of ruboxistaurin (0.25 mg/kg) and lovastatin (5.5 mg/kg). When combined, conduction velocities were within the nondiabetic range for sensory conduction, although a modest deficit remained for motor conduction. Nonetheless, interactions for both measures were highly significant (one sample t-test, observed vs predicted, p<0.0001). The trend in nerve conduction measures was continued for sciatic nerve nutritive perfusion (FIG. 5).

Blood pressure was slightly depressed in the diabetic groups, irrespective of treatment, compared to the nondiabetic control group. The drugs individually did not significantly improve nerve perfusion, although appropriate trends can be seen for group means. With joint treatment, both flow and conductance were in the nondiabetic, showing a significant synergistic interaction (one sample t-test, observed vs predicted, p=0.0015 for flow, p=0.011 for conductance).

TABLE
Ruboxistaurin - Lovastatin Interaction Study

| | | Body | Plasma | | | Blood | Blood | Vascular |
|---|---|---|---|---|---|---|---|---|
| | | wt. | Glucose | MNCV | SNCV | Flow | Pressure | Conductance |
| Group | n | (g) | (mM) | (m/s) | (m/s) | (ml/min/100 g) | (mm Hg) | (ml/min/100 g/mm Hg) |
| C | 8 | 472 ± 12 | 7.7 ± 1.0 | 64.1 ± 0.4 | 60.0 ± 0.7 | 16.1 ± 0.9 | 135.6 ± 6.3 | 0.120 ± 0.007 |
| D | 8 | 378 ± 9 | 43.2 ± 1.5 | 51.1 ± 0.7 | 49.9 ± 0.8 | 8.6 ± 0.7 | 108.1 ± 5.7 | 0.080 ± 0.004 |
| LY | 8 | 383 ± 9 | 46.4 ± 2.8 | 53.5 ± 0.3 | 54.8 ± 0.3 | 10.7 ± 0.4 | 109.8 ± 5.2 | 0.101 ± 0.008 |
| LOV | 10 | 385 ± 13 | 47.0 ± 2.2 | 54.0 ± 0.4 | 54.6 ± 0.4 | 10.4 ± 0.6 | 115.5 ± 5.2 | 0.091 ± 0.006 |
| LYLOV | 10 | 386 ± 17 | 46.6 ± 4.0 | 61.7 ± 0.5 | 60.7 ± 0.5 | 16.5 ± 0.8 | 115.1 ± 4.7 | 0.145 ± 0.010 |

Motor and sensory nerve conduction velocity (MNCV, SNCV) and the effects of ruboxistaurin (LY) or lovastatin (LOV) treatment, alone and in combination (LYLOV).
Data are mean ± SEM. Statistics, ANOVA and Neuman-Keuls multiple comparisons post-hoc test: (C): nondiabetic control group; D): diabetic control group; (LY): ruboxistaurin alone; (LOV): lovastatin alone;(LYLOV): combination of ruboxistaurin and lovastatin.

The tables show a marked synergistic interaction between ruboxistaurin and the representative HMG-CoA reductase inhibitor lovastatin. This synergy is particularly relevant in the treatment of diabetic neuropathy and vasculopathy.

1. A method of treating one or more vascular complications to hyperglycemia comprising administering to a patient in need of said treatment a therapeutically effective amount of ruboxistaurin, or a pharmaceutically acceptable salt thereof in combination with a therapeutically effective amount of an HMG-CoA reductase inhibitor.

2. The method according to claim 1 wherein said patient is a human.

3. The method according to claim 2 wherein said patient has type I or type II diabetes mellitus.

4. The method according to claim 3 wherein said vascular complication to hyperglycemia is selected from retinopathy, neuropathy, and nephropathy.

5. The method according to claim 4 wherein said patient has been diagnosed as having diabetic retinopathy.

6. The method according to claim 3 wherein said vascular complication to hyperglycemia is selected from congestive heart failure or hypertension.

7. The method according to claim 6 wherein said patient has been diagnosed as having congestive heart failure.

8. The method according to claim 5 wherein said HMG-CoA reductase inhibitor is selected from lovastatin, simvastatin, pravastatin, fluvastatin, atorvastatin, rivastatin, or a pharmaceutically acceptable salt thereof.

9. The method according to claim 8 wherein said HMG-CoA inhibitor is lovastatin; or a pharmaceutically acceptable salt thereof.

10. The method according to claim 9 wherein said ruboxistaurin or pharmaceutically acceptable salt thereof is administered in an amount of 8, 16, or 32 mg (on a ruboxistaurin basis) one to three times per day.

11. The method according to claim 10 wherein said HMG-CoA inhibitor is administered in an amount of from about 5 mg/day to about 600 mg/day.

12. The method according to claim 11 wherein said pharmaceutically acceptable salt for ruboxistaurin is the mesylate.

13-21. (canceled)

22. A pharmaceutical formulation comprising ruboxistaurin, or a pharmaceutically acceptable salt thereof; a HMG-CoA reductase inhibitor selected from the group consisting essentially of lovastatin, simvastatin, pravastatin, fluvastatin, atorvastatin, rivastatin, or a pharmaceutically acceptable salt thereof; and a pharmaceutical carrier, diluent, or excipient.

23. The pharmaceutical formulation according to claim 22 wherein said HMG-CoA reductase inhibitor is lovastatin or a pharmaceutically acceptable salt thereof.

24. The pharmaceutical formulation according to claim 23 wherein said ruboxistaurin or pharmaceutically acceptable salt thereof is present in an amount of 8, 16, or 32 mg (on a ruboxistaurin basis).

25. The pharmaceutical formulation according to claim 24 wherein said pharmaceutically acceptable salt of ruboxistaurin is the mesylate.
